(19) United States
(12) Patent Application Publication
(10) Pub. No.: US 2017/0374455 A1
(43) Pub. Date: Dec. 28, 2017

(54) MOUNTABLE SOUND CAPTURE AND REPRODUCTION DEVICE FOR DETERMINING ACOUSTIC SIGNAL ORIGIN

(71) Applicant: 3M INNOVATIVE PROPERTIES COMPANY, St. Paul, MN (US)

(72) Inventors: Mahesh C. SHASTRY, St. Paul, MN (US); Brock A. HABLE, Woodbury, MN (US); Justin TUNGJUNYATHAM, Roseville, MN (US); Jonathan T. KAHL, St. Paul, MN (US); Magnus S.K. JOHANSSON, Jönköping (SE); Abel Gladstone MANGAM, Värnamo (SE); Richard L. RYLANDER, Stillwater, MN (US)

(21) Appl. No.: 15/544,594

(22) PCT Filed: Jan. 14, 2016
(86) PCT No.: PCT/US2016/013362

§ 371 Date: Jul. 19, 2017

Related U.S. Application Data
(60) Provisional application No. 62/105,372, filed on Jan. 20, 2015.

(51) Int. Cl. H04R 3/00; H04R 29/00; A61F 11/14; H04R 1/40; G10L 25/51; H04R 1/10

(52) U.S. Cl. CPC H04R 3/005; H04R 1/406; G10L 25/51; H04R 29/00; A61F 11/14; H04R 1/1008; H04R 2430/20

(57) ABSTRACT

Sound capture and reproduction devices that can be mounted on hearing protective headsets, and are capable of using multiple microphones to determine the origins of one or more acoustic signals relative to the devices orientation, as well as methods of acquiring the origins of a combination of one or more acoustic signals from at least two microphones are described.

### FIELD

The present description relates to sound capture and reproduction devices that can be mounted on hearing protective headsets, and methods of acquiring the origins of a combination of one or more acoustic signals from two microphones.

### BACKGROUND

Hearing protection devices, including hearing protectors that include muffs worn over the ears of a user, are well known and have a number of applications, including industrial and military applications. Hearing protection devices, hearing protection headsets, and headsets are used interchangeably throughout. One common drawback of a hearing protection device is that such a device diminishes the ability of a user to identify the originating location of sound sources. This concept can be understood as spatial situational awareness. The outer ear (i.e. pinna) improves the spatial cues from binaural hearing and enhances the ability for the brain to process these cues and localize sounds. When a headset is worn, the outer ear is covered, resulting in distortion of the outer ear function. Such determination of spatial locations of sound sources is important for a user's situational awareness, whether the application is industrial or military. There exists a need to enhance the determination of the nature and location of acoustic signals for wearers of hearing protection devices.

### SUMMARY

In one aspect, the present description relates to a sound capture and reproduction device. The sound capture and reproduction device includes two microphones localized at two regions and a processor. The processor is configured to receive one or more acoustic signals from the two microphones localized at the two regions, compare the one or more acoustic signals between the two microphones, and quantitatively determine the origin of the one or more acoustic signals relative to the device orientation. The processor may be configured to receive one or more signals from the two microphones synchronously. The processor may also be configured to classify the one or more acoustic signals. The sound capture and reproduction device may also further include an orientation sensor that is capable of providing an output for determining device orientation. The processor may also be configured to receive output from the orientation sensor to determine device orientation. Additionally the device may include three or potentially four microphones, at three or four regions, respectively. In another embodiment, the device may include more than four microphones. In one embodiment, the device will be worn on the head of a user.

In another aspect, the present description relates to a method of acquiring the origins of a combination of one or more acoustic signals from two microphones. The method includes the steps of capturing the one or more acoustic signals, comparing the one or more acoustic signals between the two microphones, and quantitatively determining the origin of the one or more acoustic signals relative to the device orientation. The method may further include the steps of classifying the one or more acoustic signals and/or determining the device orientation.

### BRIEF DESCRIPTION OF THE DRAWINGS

FIG. 1 is a perspective view of a sound capture and reproduction device according to the present description.

FIG. 2 is a block diagram of a device according to the present description.

FIGS. 3A-3C are perspective views of a sound capture and reproduction device according to the present description.

FIG. 4 is a flow chart of a method of acquiring the origins of a combination of one or more acoustic signals from two microphones.

FIG. 5 illustrates a coordinate system used in characterizing a wave vector.

FIG. 6 is a flow chart illustrating a method of acquiring the origins of acoustic signals.

FIG. 7 is a block diagram of a sub-system that implements estimation of a generalized cross-correlation function used in determining acoustic signal location.

FIG. 8 is a block diagram of a cross-correlation function that estimates angle of direction of arrival of acoustic signals based on inputs of time-differences of arrival.

FIG. 9 is a graph illustrating actual vs. estimated angle of arrival with different microphone combinations.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

In the following detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example.

As noted above, currently used headsets suffer the common drawback of diminished ability of a user to identify the originating location of sound sources, due to the covering of the outer ears and their ability to aid in spatial cues for the brain's processing of sound localization. There therefore exists a need to enhance determination and localization of acoustic signals for wearers of hearing protection devices. The present description provides a solution to this need, and a means to enhance spatial situational awareness of users of hearing protection devices.

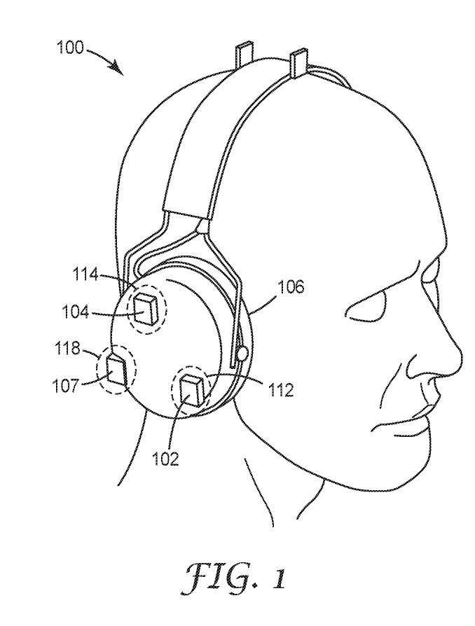

FIG. 1 provides a perspective view of a sound capture and reproduction device 100 according to the present description. As illustrated in FIG. 1, in one embodiment, the sound capture and reproduction device may be worn on the head of a user, e.g., as part of a hearing protection device with protective muffs provided over the ears of a user. Reproduction, as used throughout this disclosure, may refer to the reproduction of the sound source location information, such as audible, visual and haptic feedback. Sound capture and reproduction device 100 includes at least two microphones. The device includes first microphone 102 positioned in a first region of the device 112. Additionally the device includes second microphone 104 positioned in a second region of the device 114. First microphone 102 and second microphone 104 are generally positioned at two regions (112, 114) that are optimal for accurately determining the origin of the one or more acoustic signals. An exemplary microphone that may be used as the first and second microphones 102, 104 is the INMP401 MEMS microphone from Invensense of San Jose, Calif.

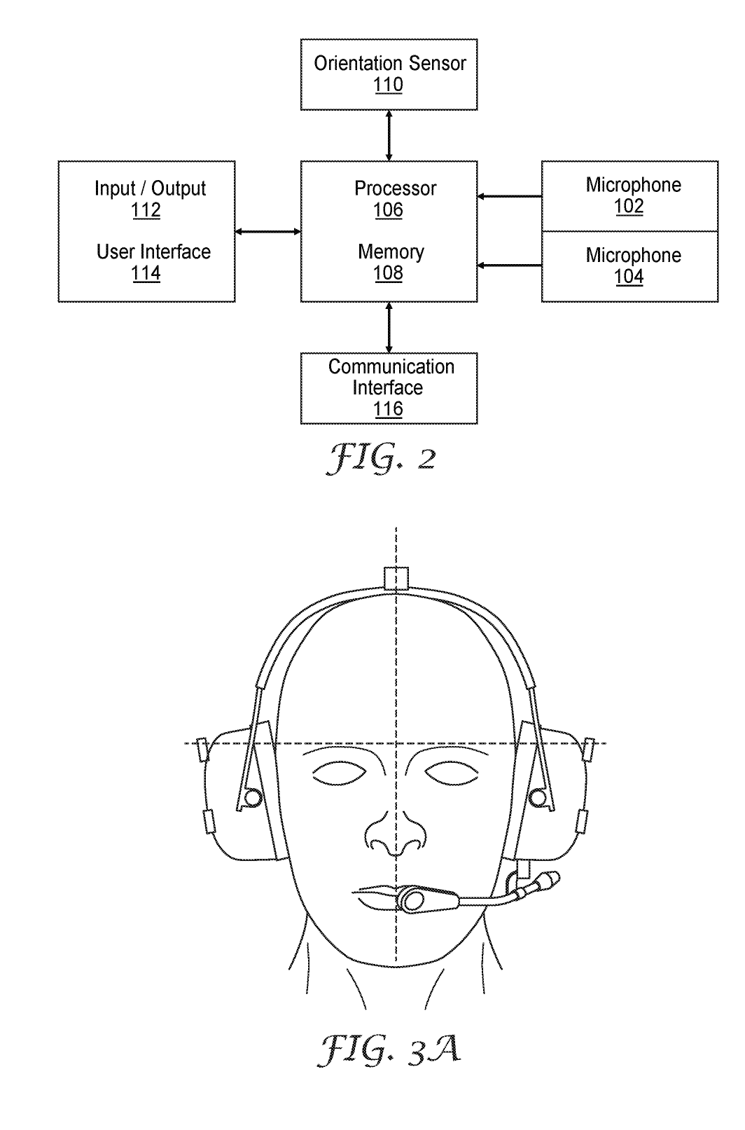

Sound capture and reproduction device 100 further includes a processor 106 that can be positioned within the ear muff, in the headband of the device, or in another appropriate location. Processor 106 is configured to perform a number of functions using input acquired from the microphones 102, 104. The processor is configured to receive the one or more acoustic signals from the two microphones (first microphone 102 and second microphone 104) and compare the one or more acoustic signals between the two microphones. Utilizing this comparison, the processor 106 is capable of quantitatively determining information about the origin of the one or more acoustic signals relative to the device orientation. This quantitative determination of the acoustic signals, including computation of the origin, can include, e.g., measurements of azimuth, elevation, distance or spatial coordinates of the signals. A better understanding of the system may be gained by reference to the block diagram in FIG. 2.

The processor 106 may include, for example, one or more general-purpose microprocessors, specially designed processors, application specific integrated circuits (ASIC), field programmable gate arrays (FPGA), a collection of discrete logic, and/or any type of processing device capable of executing the techniques described herein. In some embodiments, the processor 106 (or any other processors described herein) may be described as a computing device. In some embodiments, the memory 108 may be configured to store program instructions (e.g., software instructions) that are also executed by the processor 106 to carry out the processes or methods described herein. In other embodiments, the processes or methods described herein may be executed by specifically programmed circuitry of the processor 106. In some embodiments, the processor 106 may thus be configured to execute the techniques for acquiring the origins of a combination of one or more acoustic signals described herein. The processor 106 (or any other processors described herein) may include one or more processors. Processor may further include memory 108. The memory 108 stores information. In some embodiments, the memory 108 can store instructions for performing the methods or processes described herein. In some embodiments, sound signal data may be pre-stored in the memory 108. One or more properties from the sound signals, for example, category, phase, amplitude, and the like may be stored as the material properties data.

The memory 108 may include any volatile or non-volatile storage elements. Examples may include random access memory (RAM) such as synchronous dynamic random access memory (SDRAM), read-only memory (ROM), non-volatile random access memory (NVRAM), electrically erasable programmable read-only memory (EEPROM), and FLASH memory. Examples may also include hard-disk, magnetic tape, a magnetic or optical data storage media, and a holographic data storage media.

The processor 106 may, in some embodiments, be configured to receive the one or more acoustic signals from the two microphones synchronously. Acquiring synchronized acoustic signals permits accurate and expeditious analysis as the time and resources required for the processor 106 to align or correlate the data prior to determination of the sound source origin are minimized. Synchronization maintains data integrity, coherence, and format enabling repeatable acquisition, consistent comparison, and precise computations. The one or more acoustic signals may be synchronized with respect to frequency, amplitude, phase, or wavelength. Where the processor 106 receives acoustic signals synchronously, in some embodiments, it may receive those signals simultaneously, while in others the processor will receive the signals sequentially. Simultaneous reception is advantageous in that the method for determining the origin of the sound source may immediately begin upon acquisition and transmission to the processor 106.

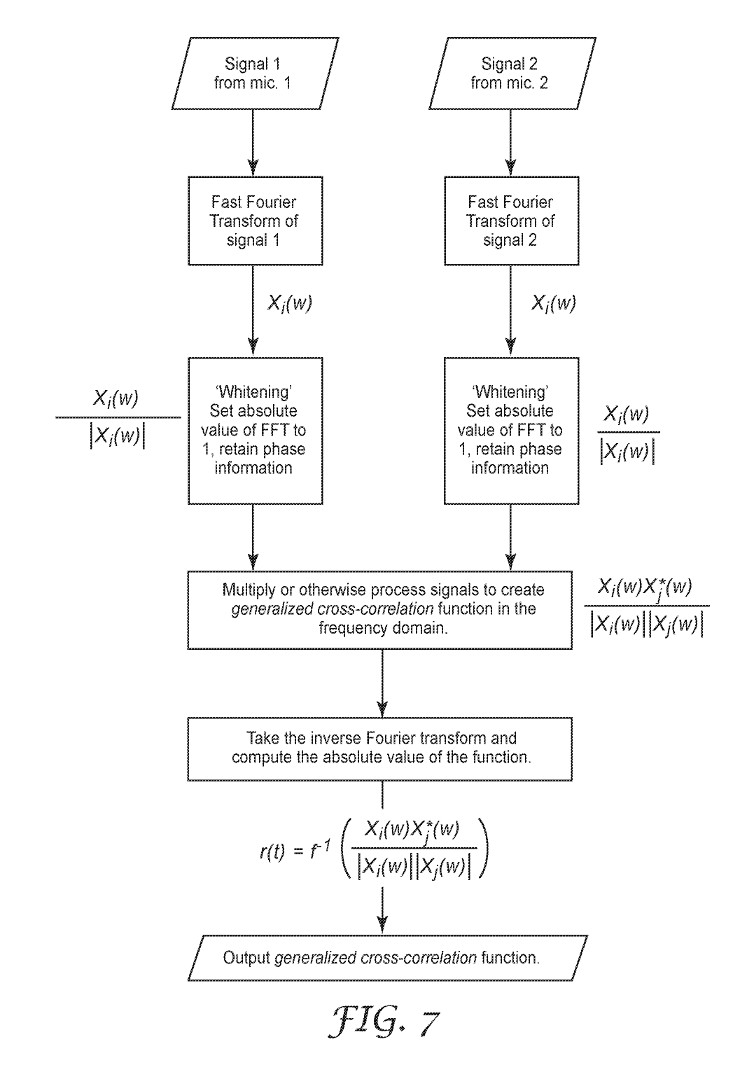

In at least one embodiment, the processor 106 may further be configured to classify the one or more acoustic signals received. Classifying the acoustic signal or signals may include identifying whether the signal belongs to one or more categories, including: background noise, speech and impulse sounds. In one embodiment, the processor may be configured to compare the one or more acoustics signals based upon classification between the two microphones in a pairwise manner as described further in FIG. 7.

The sound capture and reproduction device 100 of the present description may further include input/output device 112 and user interface 114 to provide visual, audible, haptic, or tactile feedback about sound source location. Where the feedback is audible the means of providing the feedback may be a loudspeaker. Where the feedback is visual, the feedback may be, e.g., blinking lights located in view of a user.

Input/output device 112 may include one or more devices configured to input or output information from or to a user or other device. In some embodiments, the input/output device 112 may present a user interface 114 where a user may define operation and set categories for the sound capture and reproduction device. For example, the user interface 114 may include a display screen for presenting visual information to a user. In some embodiments, the display screen includes a touch sensitive display. In some embodiments, a user interface 114 may include one or more different types of devices for presenting information to a user. The user interface 114 may include, for example, any number of visual (e.g., display devices, lights, etc.), audible (e.g., one or more speakers), and/or tactile (e.g., keyboards, touch screens, or mice) feedback devices. In some embodiments, the input/output devices 112 may represent one or more of a display screen (e.g., a liquid crystal display or light emitting diode display) and/or a printer (e.g., a printing device or component for outputting instructions to a printing device). In some embodiments, the input/output device 112 may be configured to accept or receive program instructions (e.g., software instructions) that are executed by the processor 106 to carry out the embodiments described herein.

The sound capture and reproduction device 100 may also include other components and the functions of any of the illustrated components including the processor 106, the memory 108, and the input/output devices 112 may be distributed across multiple components and separate devices such as, for example, computers. The sound capture and reproduction device 100 may be connected as a workstation, desktop computing device, notebook computer, tablet computer, mobile computing device, or any other suitable computing device or collection of computing devices. The sound capture and reproduction device 100 may operate on a local network or be hosted in a Cloud computing environment.

The sound capture and reproduction device may additionally include an orientation sensor 110. The orientation sensor 110 is capable of providing an output for determining device orientation relative to the environment in which the device is operating. Although it may be mounted on the muff, the orientation sensor 110 may be mounted at any appropriate position on the sound capture and reproduction device that allows it to properly determine device orientation (e.g. on the headband between the muffs). In one embodiment, the orientation sensor 110 may include an accelerometer. In another embodiment, the orientation sensor 110 may include a gyroscope. Alternatively, the orientation sensor 110 may include a compass. In some embodiments, a combination, or all three of these elements may make up the orientation. In some embodiments, the orientation sensor 110 will be capable of providing reference points for localization. Examples of orientation sensors 110 may include the ITG-3200 Triple-Axis Digital-Output Gyroscope from Invensense of San Jose, Calif., the ADXL345 Triple-axis Accelerometer from Analog Devices of Norwood, Mass., or the HMC5883L Triple Axis Digital Magnetometer from Honeywell of Morrisville, N.J.

Communication interface 116 may be a network interface card, such as an Ethernet card, an optical transceiver, a radio frequency transceiver, or any other type of device that can send and receive information. Other examples of such communication interfaces may include Bluetooth, 3G, 4G, and WiFi radios in mobile computing devices as well as USB. In some examples, sound capture and recording device 100 utilizes communication interface 116 to wirelessly communicate with external devices such as a mobile computing device, mobile phone, workstation, server, or other networked computing device. As described herein, communication interface 116 may be configured to receive sounds signal categories, updates, and configuration settings as instructed by processor 106.

Where the sound capture and reproduction device 100 of the present description is positioned on a headset having protective ear muffs, the microphones 102, 104 (and potentially others, where applicable) may be integrated with sound control capabilities. Sound control capabilities can include the ability to filter, amplify, attenuate and sound received by microphones 102 and 104. Additionally, the protective muff may have at least a certain passive noise reduction or sound attenuation, and a microphone disposed exteriorly on the hearing protection device, a loudspeaker disposed in the muff, and an amplifier for amplifying acoustic signals received by the microphone and passing the signals onto the loud speaker, such as described in commonly owned and assigned PCT Publication No. WO 2006/058319, which is hereby incorporated by reference in its entirety. In such an embodiment, the loudspeaker is capable of not transmitting signals received by the microphone that are above a certain decibel level or sound pressure level or correspond to impulse events (e.g. gunshots, or loud machinery noises).

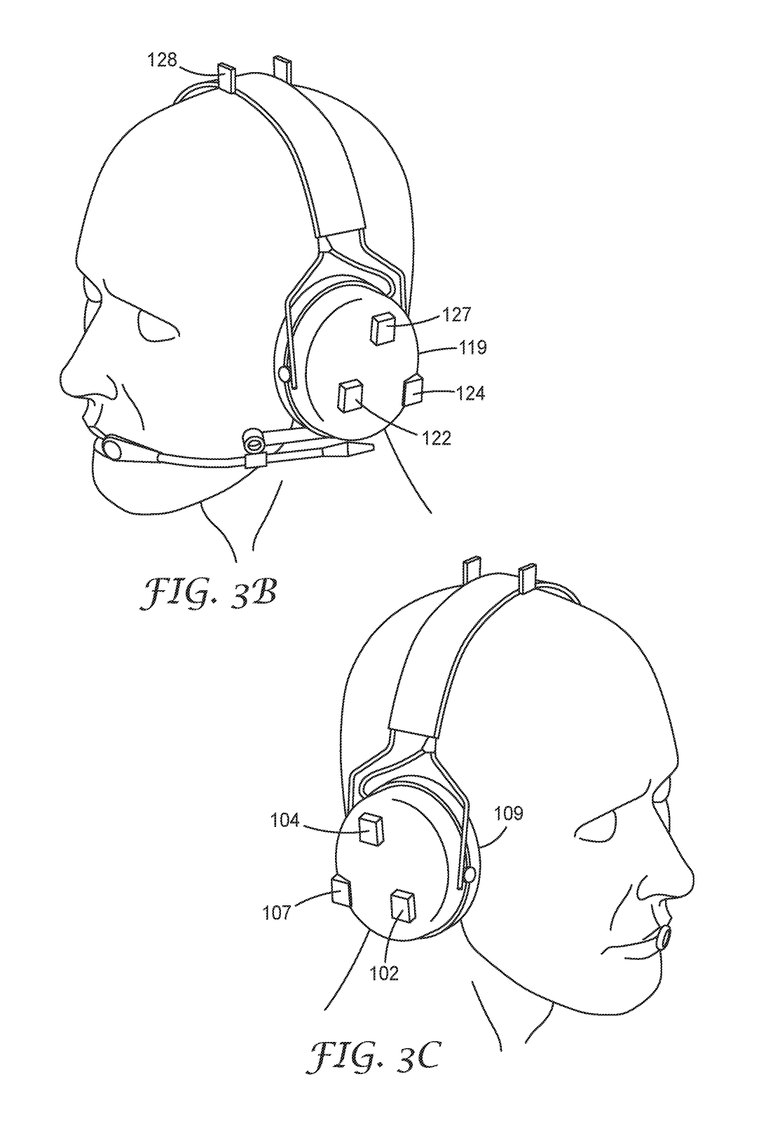

Sound capture and reproduction device 100 may include more than two microphones that feed information to the processor 106. For example, the device may include a third microphone 107, located at a third region 118, where each of the three regions 112, 114 and 118 are optimally localized for most effective determination of acoustic signal localization. In such a case, the processor 106 will receive and compare acoustic signals between all three microphones. Alternatively the device may include four microphones optimally localized at four regions, where the processor receives and compares acoustic signals between all four microphones. In fact, the device can include any other appropriate number of microphones, e.g., five, six, seven, eight or more, as a greater number of microphones will aid in greater accuracy as to location of sound. Microphones described herein may, in some embodiments include omnidirectional microphones (i.e. microphones picking up sound from all directions). However, to aid in localization of sound sources, and improve the difference of the signal between microphones, directional microphones may be used, or mechanical features can be added near a given microphone region to focus or diffuse sounds coming from specific directions. FIGS. 3A-3C represent an embodiment having first, second and third microphones 102, 104 and 107, on a first protective muff 109, fourth, fifth and sixth microphones 122, 124 and 127 on a second protective muff 119 and a seventh microphone 128 on the headband connecting first and second protective muffs.

In another aspect, the present description relates to a method of acquiring the origins of a combination of one or more acoustic signals from two microphones. The method, as illustrated by the flowchart in FIG. 4 includes the steps of: capturing the one or more acoustic signals (301), comparing the one or more acoustic signals from two microphones (302), and quantitatively determining the origin of the one or more acoustic signals relative to the device orientation (303). The steps of comparing the signals and quantitatively determining their origin may, in some embodiments, be performed using a processor, such as processor 106 described above. Though not shown in FIG. 4, the method may include the further step of classifying the one or more acoustic signals, such as in the manner discussed above and with respect to FIG. 7. The method may also include the step of determining device orientation using, e.g., an orientation sensor 110.

Additionally, the method may be a method of acquiring the origins of a combination of one or more acoustic signals from three, four, five or more microphones, in which case sound signals from each of the microphones are compared by the processor.

The mathematical methodology by which the processor is able to localize sound by comparing the acoustic signal or signals from various microphones at different locations relates to comparing the phase shifts of acoustic signals received from the two or more microphones using the processor. To describe in further detail the function of the system mathematically, we may introduce the following defined elements in Table 1:

TABLE 1

| Symbol | Definition |
|---|---|
| r | [x, y, z] |
| a(r, t) | Amplitude of sound wave |
| k | Wave vector |
| rᵢ | [kₓ, k_{y}, k_{z}] |
| a(rᵢ, t) | Amplitude of sound wave at location rᵢ |
| xᵢ(t) | Time series of sound wave at microphone i |
| τᵢⱼ | Time difference of arrival between microphone i and |
| | microphone j |
| F | Fourier transform operator |
| D | Microphone location difference |

The equation of a wave coming in at an arbitrary direction from a source located at the spherical co-ordinates (R, θ, φ) is given by Equation 1,

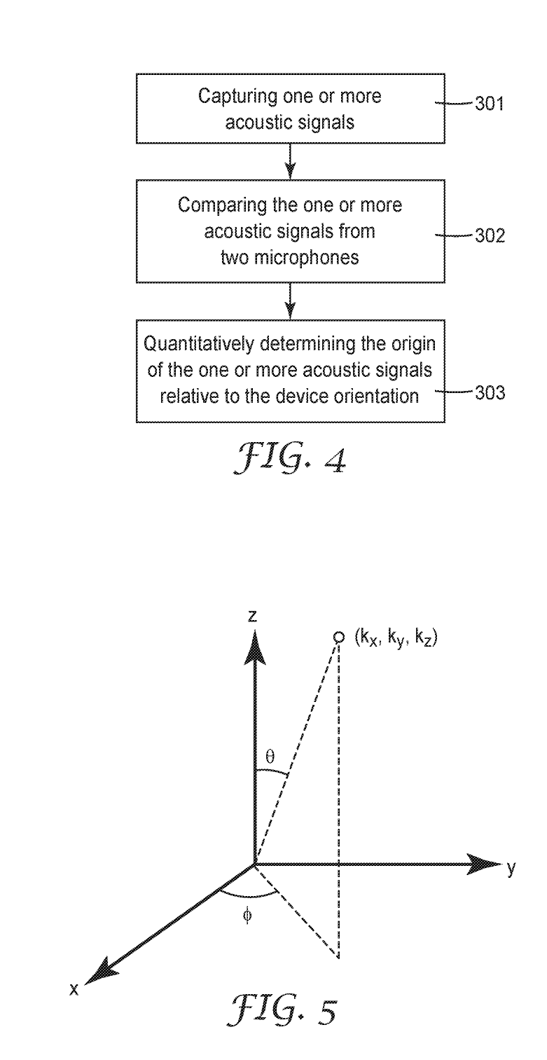

a(r,t)=A₀e^{-i(k·r+ωt)} Equation 1:

where k is the wave vector, which is an extension of the wave number to waves propagating in arbitrary direction in space. Let the location of each microphone (indexed by i) be denoted by the vector representing its Cartesian coordinates, rᵢ=[xᵢ, yᵢ, zᵢ]. An illustration of such a coordinate system is provided in FIG. 5. The wave measured by each microphone is then given by Equation 2,

aᵢ(rᵢ,t)=A₀e^{-i(k·r}^{ᵢ}^{+ωt)}. Equation 2:

The sound waves arriving at different microphones are delayed with respect to one another. The phase difference between two microphones (indexed by i and j), is given by Equation 3,

τᵢⱼ=k^{T}(rᵢ−rⱼ) Equation 3:

If we have an N-microphone array, there are N(N−1)/2 microphone-pairs.

$$\begin{matrix}r ( τ ) = \int {x}_{i} ( t + τ ) {x}_{i} ( t ) dt & Equation 4 \\ r ( τ ) = {F}^{- 1} ( \frac{{X}_{i} ( ω ) {X}_{j}^{*} ( ω )}{\vert {X}_{i} ( ω ) \vert \vert {X}_{j} ( ω ) \vert} ) & Equation 5 \\ {τ}_{ij} = {argmax}_{τ} \vert r ( τ ) \vert & Equation 6 \\ {a}_{i} ( {r}_{i} , t ) = {A}_{0} {e}^{\{ - i ( k {r}_{i} + ω t ) \}} & Equation 7 \\ {τ}_{12} = {k}^{T} ( {r}_{1} - {r}_{2} ) & Equation 8 \\ {τ}_{N ( N - 1 )} = {k}^{T} ( {r}_{N} - {r}_{N - 1} ) & Equation 9 \\ τ = Dk & Equation 10 \\ k = {( {D}^{T} D )}^{- 1} {D}^{T} τ & Equation 11 \\ τ = [ \begin{matrix}{τ}_{12} \\ ⋮ \\ {τ}_{N - 1 , N}\end{matrix} ] & Equation 12 \\ D = [ \begin{matrix}{x}_{2} - {x}_{1} & {y}_{2} - {y}_{1} & {z}_{2} - {z}_{1} \\ ⋮ & ⋱ & ⋮ \\ {x}_{N -} {x}_{N - 1} & {y}_{N -} {y}_{N - 1} & {z}_{N} - {z}_{N - 1}\end{matrix} ] & Equation 13 \\ k = [ \begin{matrix}{k}_{x} \\ {k}_{y} \\ {k}_{z}\end{matrix} ] & Equation 14 \\ Azimuthal angle \text{:} φ = arccos \frac{{k}_{y}}{\sqrt{( {k}_{x}^{2} + {k}_{y}^{2} )}} & Equation 15 \\ Elevation angle \text{:} θ = arccos \frac{{k}_{z}}{\sqrt{( {k}_{x}^{2} + {k}_{y}^{2} + {k}_{z}^{2} )}} & Equation 16\end{matrix}$$

If two or more microphones are collinear, then Equation 10, reduces to a scalar equation with the solution being:

$$k = \frac{{τ}_{12}}{( {X}_{2} - {X}_{1} )} \qquad Equation 17$$

The ambiguous angle of the sound source would be:

$$∅ = arccos \frac{{τ}_{12}}{c ( {X}_{2} - {X}_{1} )} \qquad Equation 18$$

A unique k is observed if the microphones are non-coplanar. Three microphones are always coplanar. It could also be that there are more than three microphones, but they are all located in a single plane. In such a case, the system may be solved, but it will result in multiple solutions for the variable k. The solution would then imply that the sound source is located at a particular angle on either side of the plane defined by the microphones. The solution would be:

$$\begin{matrix}k = {( {D}^{T} D )}^{- 1} {D}^{T} τ & Equation 19 \\ τ = [ \begin{matrix}{τ}_{12} \\ ⋮ \\ {τ}_{N - 1 , N}\end{matrix} ] & Equation 20 \\ D = [ \begin{matrix}{x}_{2} - {x}_{1} & {y}_{2} - {y}_{1} \\ ⋮ & ⋮ \\ {x}_{N -} {x}_{N - 1} & {y}_{N -} {y}_{N - 1}\end{matrix} ] & Equation 21 \\ k = [ \begin{matrix}{k}_{x} \\ {k}_{y}\end{matrix} ] & Equation 22 \\ Azimuthal angle \text{:} φ = arccos \frac{{k}_{y}}{\sqrt{( {k}_{x}^{2} + {k}_{y}^{2} )}} & Equation 23 \\ Elevation angle \text{:} θ is undetermined . & Equation 24\end{matrix}$$

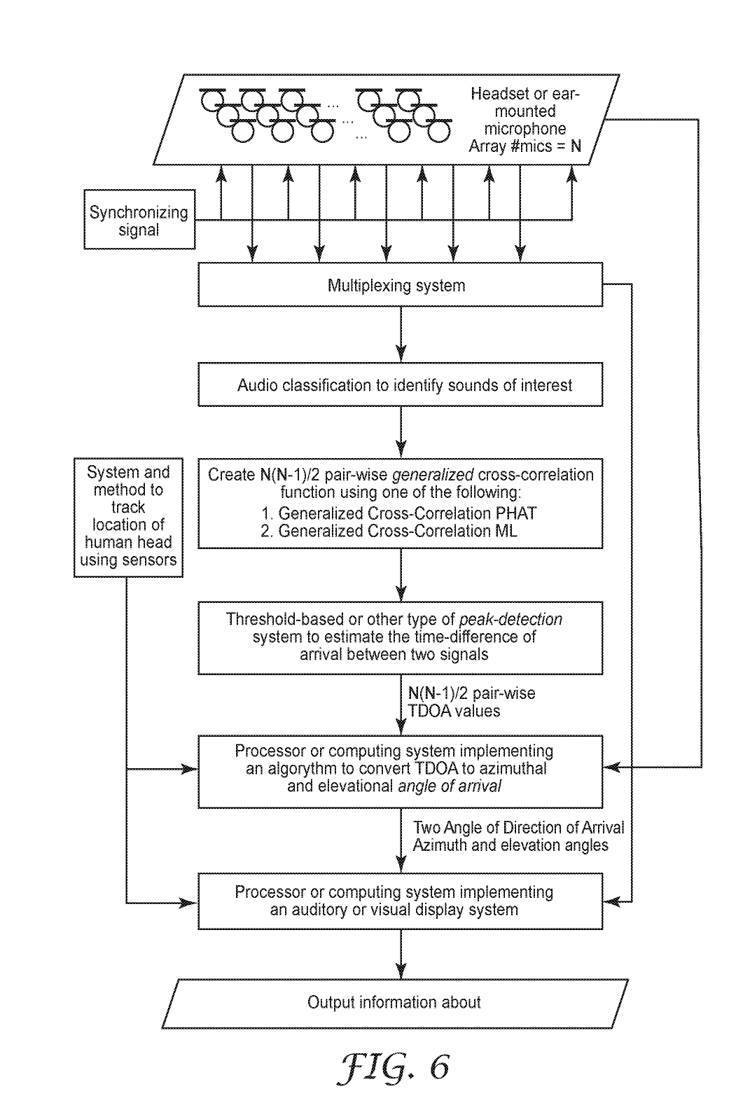

A system consisting of at least 4 microphones and at least one microphone that is not in the same plane as the others would result in three variables present in the equations. However, any three microphones define a plane. In order to overcome this problem, information from a fourth non-planar microphone is needed so that det(D^{T}D)≠0, which is to say that D is non-singular. Thus, mathematically, the preferred mode for unambiguous and robust computation of 3D angles would be to include at least four microphones as represented in Equations 10-16. A flow chart illustrating a method of acquiring the origins of acoustic signals as described above is illustrated in FIG. 6.

### EXAMPLES

### Example 1

Applicants created a sound capture and reproduction device as part of a hearing protection device containing two protective muffs and a headband connecting the muffs. Three INMP401 MEMS microphones from Invensense of San Jose, Calif. were arranged in a triangle arrangement on each on the two protective muffs. Additionally, two INMP401 MEMS microphones from Invensense of San Jose, Calif. were positioned on the headband. The coordinates and location of each microphone is provided in Table 2:

TABLE 2
Microphone Coordinates

| | Mic 1 | Mic 2 | Mic 3 | Mic 4 | Mic 5 | Mic 6 | Mic 7 | Mic 8 |
|---|---|---|---|---|---|---|---|---|
| (meters) | (LF) | (LT) | (LB) | (RF) | (RT) | (RB) | (TF) | (TB) |
| x | 0.0254 | 0 | −0.0254 | 0.0254 | 0 | −0.0254 | 0.0254 | −0.0254 |
| y | 0.1016 | 0.1016 | 0.1016 | −0.1016 | −0.1016 | −0.1016 | 0 | 0 |
| z | −0.0861 | 0 | −0.0861 | −0.0861 | 0 | −0.0861 | 0.1016 | 0.1016 |

where:

LF=Left Front, LT=Left Top, LB=Left Back, RF=Right Front, RT=Right Top, RB=Right Back, TF=Top Front and TB=Top Back.

The eight-microphone array provided flexibility to perform subsets of measurements and determine which microphone configurations gave good localization performance. The microphone array headset was placed on a 45BB KEMAR Head & Torso, non-configured manikin from G.R.A.S Sound and Vibration of Holte, Denmark. A BOSE® Soundlink wireless speaker from Bose® of Framingham, Mass. was positioned approximately 5 m away for use as a sound source. The elevation angle between the 45BB KEMAR Head & Torso, non-configured manikin and the sound source was held constant at 0 or near 0 degrees. During the test the 45BB KEMAR Head & Torso, non-configured manikin head was rotated along the azimuth angle from 0 to 360 degrees. The microphones were connected to an NI USB-6366 DAQ module from National Instruments of Austin, Tex. The acquisition of the sound signals occurred simultaneously with the eight different microphone channels with 100 kHz sampling rate for each channel.

LabVIEW (from National Instruments, Austin, Tex.) software was used as an interface to acquire and post-process the acoustic signals from the channels. During post-processing the LabVIEW software computed pair-wise generalized cross-correlation functions (GCC) and determined the global maximum peak of the GCC to determine the time-difference of arrival (TDOA). The TDOA was then passed into a process block which implemented a method for estimating the angle of arrival of the acoustic waves at the microphone array.

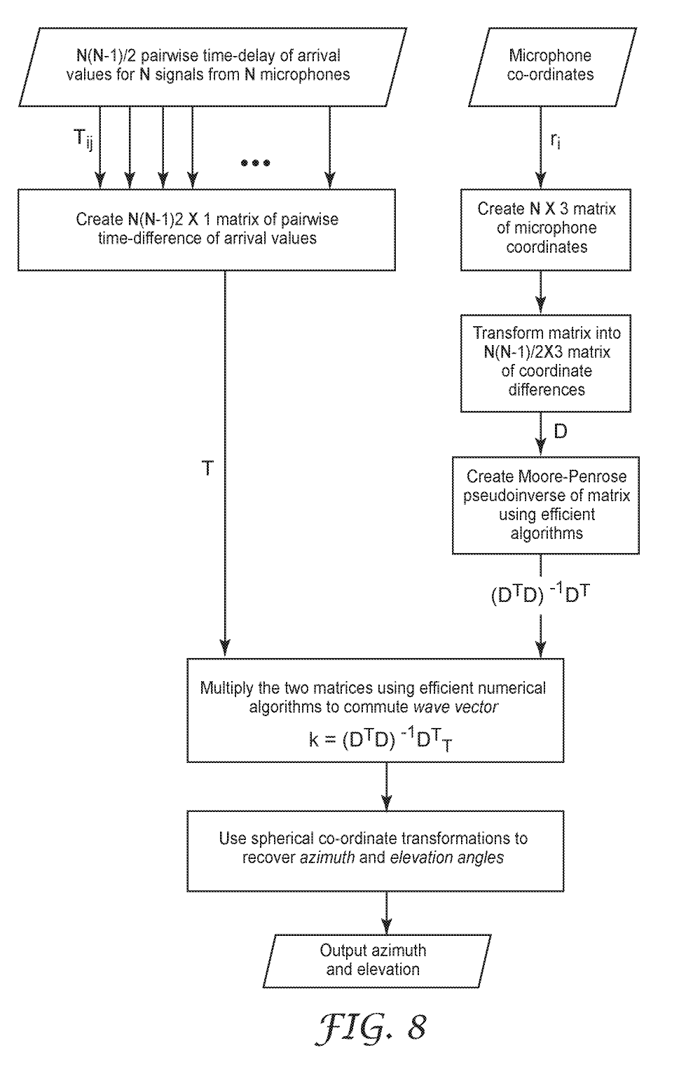

FIG. 6 provides a block diagram of a more detailed example of a method utilized for determining origins of acoustic signals. The input to the example consists of sound pressure variation caused by airborne sound waves recorded at multiple microphones. The analog signals are converted to digital signals by using synchronized analog to digital converters (ADCs). The ADCs can be integrated into the microphones or are external to the microphone transducer system. The ADCs are all synchronized by a synchronizing signal. The signals from these multiple channels are multiplexed for processing on an embedded processor, digital signal processor, or computing system. The synchronized and multiplexed signals are processed pairwise to, for example, compute the angle generalized cross-correlation function. The generalized cross-correlation function is illustrated in FIG. 7. The generalized cross-correlation function (GCC) is input into a sub-system that finds the global maximum peak of the GCC to compute the time-difference of arrival. The time-difference of arrival of the signal is then passed into a processor which implements a method for estimating the angle of arrival of the sound waves at the microphone array as shown in FIG. 8. The last stage involves a processor implementing an auditory or visual display system to alert the user to the direction of the sound source.

FIG. 8 illustrates a block diagram of the use of a generalized cross-correlation function that takes as inputs the time-differences of arrival and estimates the angle of direction of arrival. The pairwise time-differences of arrival and the microphone coordinates are input into a sub-system that computes the angle of arrival of the sound waves using algorithms such as the one shown in FIG. 8. The time distance of arrival matrix is constructed based on the N(N−1)/2 pairwise time-differences of arrival, where N is the number of microphones.

### Example 2

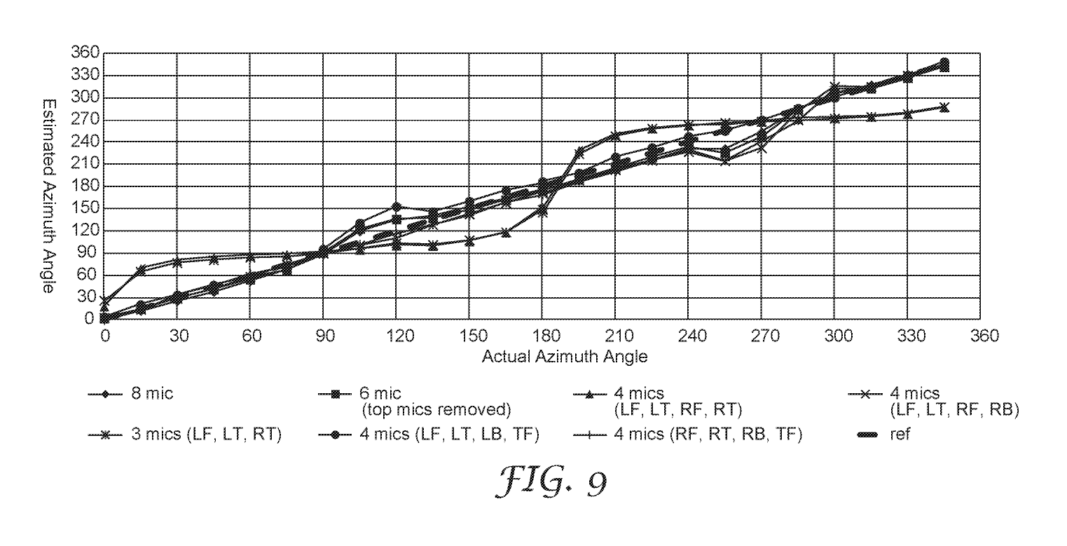

Following Example 1, and the methods disclosed above, Applicants tested a number of different microphone number and position combinations. The results of the testing are illustrated in FIG. 9, a graph mapping Actual vs. Estimated Angle of Arrival with Different Microphone Combinations. Based on the results shown, the four-microphone configurations with non-symmetrical arrangements on each side of the headset (LF-LT and RF-RB) provided good results when compared to the eight microphone case. It was determined that another good arrangement for the azimuth localization included three microphones on one side of a headset (e.g. on one muff) and one either on the top the headband or on the opposite side of the headset. This arrangement provided advantages in minimizing the geometry calibration, i.e. fixed distance between microphones since most were located on the one side.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A sound capture and reproduction device, comprising:
two microphones localized at two regions; and
a processor, wherein the processor is configured to:
receive one or more acoustic signals from the two microphones localized at two regions,
compare the one or more acoustics signals between the two microphones, and
quantitatively determine the origin of the one or more acoustic signals relative to the device orientation.

2. The sound capture and reproduction device of claim 1, wherein the processor is configured to receive one or more signals from the two microphones synchronously.

3. The sound capture and reproduction device of claim 2, wherein the processor is configured to receive one or more signals from the two microphones simultaneously.

4. The sound capture and reproduction device of claim 2, wherein the processor is configured to receive one or more signals from the two microphones sequentially.

5. The sound capture and reproduction device of claim 1, wherein the two microphones are positioned at two optimal regions for accurate determination of origin of the one or more acoustic signals.

6. The sound capture and reproduction device of claim 1, wherein the processor is configured to compare the one or more acoustics signals based upon classification between the two microphones in a pair-wise manner.

7. The sound capture and reproduction device of claim 1, further comprising an orientation sensor, the orientation sensor being capable of providing an output for determining device orientation.

8. The sound capture and reproduction device of claim 7, wherein the orientation sensor comprises an accelerometer.

9. The sound capture and reproduction device of claim 7, wherein the orientation sensor comprises a gyroscope.

10. The sound capture and reproduction device of claim 7, wherein the orientation sensor comprises a compass.

11. The sound capture and reproduction device of claim 7, wherein the orientation sensor is capable of providing reference points for localization.

12. The sound capture and reproduction device of claim 1, wherein the two microphones are integrated with sound control capabilities.

13. The sound capture and reproduction device of claim 1, wherein quantitative determinations of the one or more acoustic signals may include measurements of azimuth, elevation, distance or spatial coordinates.

14. The sound capture and reproduction device of claim 1, wherein the processor is further configured to classify the one or more acoustic signals.

15. The sound capture and reproduction device of claim 14, wherein classifying the one or more acoustic signals comprises identifying whether the signal belongs to one of the following categories: background noise, speech, and impulse sounds.

16-23. (canceled)

24. A method of acquiring the origins of a combination of one or more acoustic signals from two microphones, comprising the steps of capturing the one or more acoustic signals, comparing the one or more acoustic signals from two microphones, and quantitatively determining the origin of the one or more acoustic signals relative to the device orientation.

25. The method of claim 24 comprising the further step of classifying the one or more acoustic signals.

26. The method of claim 25, wherein classifying the one or more acoustic signals comprises identifying whether the signal belongs to one of the following categories: background noise, speech, and impulse sounds.

27. The method of claim 24 comprising the further step of determining device orientation.

28. The method of claim 27, wherein the device orientation is determined using an orientation sensor.

29-35. (canceled)
